(19) United States
(12) Patent Application Publication
(10) Pub. No.: US 2024/0182957 A1
(43) Pub. Date: Jun. 6, 2024

(54) METHODS AND DEVICES FOR NUCLEIC ACID DETECTION

(71) Applicant: The Regents of the University of Colorado, a Body Corporate, Aurora, CO (US)

(72) Inventor: Clifton Manning Carey, Denver, CO (US)

(21) Appl. No.: 18/279,769

(22) PCT Filed: Mar. 7, 2022
(86) PCT No.: PCT/US2022/019086

§ 371 Date: Sep. 1, 2023

Related U.S. Application Data
(60) Provisional application No. 63/160,141, filed on Mar. 12, 2021.

(51) Int. Cl. C12Q 1/6844; C12Q 1/04; C12Q 1/6888; G01N 33/58

(52) U.S. Cl. CPC C12Q 1/6844; C12Q 1/04; C12Q 1/6888; G01N 33/582

(57) ABSTRACT

A modified method for detecting the presence or absence of a target nucleic acid includes contacting a sample suspected of containing the target nucleic acid with one or more primer sets to amplify a portion of the target nucleic acid, divalent ions, dNTPs, a buffer, a polymerase, and an amino-acid functionalized perylene-3,4:9,10-tetracarboxylic dianhydride (PDI) dye complexed with copper (X-PDI-Cu) to form an amplification mixture, wherein amino acid X is selected from aspartic acid, glycyl-1-aspartic acid, and glutamic acid; incubating the amplification mixture under conditions to perform an amplification reaction providing amplified target nucleic acid and pyrophosphate; and detecting uncomplexed X-PDI in the reaction mixture, wherein uncomplexed X-PDI indicates the production of Cu-pyrophosphate and the presence of the target nucleic acid in the sample.

### CROSS-REFERENCE TO RELATED APPLICATIONS

This application claims priority to U.S. Provisional Application 63/160,141 filed on Mar. 12, 2021, which is incorporated herein by reference in its entirety.

### FIELD OF THE DISCLOSURE

The present disclosure is related to methods and devices for detection of nucleic acid amplification, specifically viral genes amplified from SARS-CoV-2 genome.

### BACKGROUND

Severe acute respiratory syndrome coronavirus 2 (SARS-CoV-2) is the virus that causes coronavirus-19 (COVID-19), which is responsible for a global pandemic. Since 2020, fast and accurate detection of SARS-CoV-2 infection has been an urgent global need.

Current Reverse Transcription Loop-Mediated Isothermal Amplification (RT-LAMP) technology has been used to detect COVID-19 virions in samples that are homogeneous and of low buffer capacity at pH approximately 8. RT-LAMP results in a high degree of amplification, with DNA being amplified 10⁹-10¹⁰ times in 15-60 minutes. Because of the high specificity associated with the primers used, the presence of a target gene sequence can easily be detected just by judging the presence of amplified products (DNA) or the increase in concentration of other reaction products such as H⁺ (acid) or pyrophosphate. Typically, detection is based on the change in pH of the sample using pH indicators such as phenol red which detects H⁺ ions produced, as ions are added to DNA. A significant problem with using this approach with salivary samples is that salivary samples have variable pHs, such as pH changes even within a single day, which can confound the results.

Other approaches to detection in RT-LAMP assays include fluorescent dyes such as SYBR™ Green which binds to double stranded DNA, alizarin red which can detect concentration changes in magnesium, hydroxynaphthol blue which responds to changes in magnesium concentration, and turbidity assays which measure magnesium-pyrophosphate precipitation. All of these detection methods have sensitivity limitations and also require laboratory support for the instrumentation to achieve the high temperature cycles that are part of the analysis.

What is needed are improved nucleic acid-based detection assays that overcome may of the issues with these prior art assays.

### BRIEF SUMMARY

In an aspect, a modified method for detecting the presence or absence of a target nucleic acid comprises contacting a sample suspected of containing the target nucleic acid with one or more primer sets to amplify a portion of the target nucleic acid, divalent ions, dNTPs, a buffer, a polymerase, and an amino-acid functionalized perylene-3,4:9,10-tetracarboxylic dianhydride (PDI) dye complexed with copper (X-PDI-Cu) to form an amplification mixture, wherein amino acid X is selected from aspartic acid, glycyl-1-aspartic acid, and glutamic acid; incubating the amplification mixture under conditions to perform an amplification reaction providing amplified target nucleic acid and pyrophosphate; and detecting uncomplexed X-PDI in the reaction mixture, wherein uncomplexed X-PDI indicates the production of Cu-pyrophosphate and the presence of the target nucleic acid in the sample.

In another aspect, a modified method for detecting the presence or absence of SARS CoV-2 comprises contacting a sample suspected of containing SARS CoV-2 with one or more primer sets to amplify a portion of the SARS CoV-2 genome, divalent ions, dNTPs, a buffer, a polymerase, and an amino-acid functionalized perylene-3,4:9,10-tetracarboxylic dianhydride (PDI) dye complexed with copper (X-PDI-Cu) to form an amplification mixture, wherein amino acid X is selected from aspartic acid, glycyl-1-aspartic acid and glutamic acid; incubating the amplification mixture under conditions to perform an amplification reaction providing amplified SARS CoV-2 nucleic acids and pyrophosphate; and detecting uncomplexed X-PDI in the reaction mixture, wherein uncomplexed X-PDI indicates the production of Cu-pyrophosphate and the presence of the SARS CoV-2 nucleic acids in the sample.

In an aspect, a kit comprises a reagent compartment comprising one or more primer sets to amplify a portion of a target nucleic acid, divalent ions, dNTPs, a buffer, a polymerase, and an amino-acid functionalized perylene-3,4:9,10-tetracarboxylic dianhydride (PDI) dye complexed with copper (X-PDI-Cu), wherein amino acid X is selected from aspartic acid, glycyl-1-aspartic acid and glutamic acid; and a sample collection device for collecting a sample from a subject; wherein, upon contacting the sample with the reagent compartment, an amplification reaction provides amplified target nucleic acid and pyrophosphate.

### BRIEF DESCRIPTION OF THE DRAWINGS

FIG. 1 shows that quenching is proportional to the amount of copper complexation. In this example the, ADPI concentration is 25 μmol/L and copper concentration is as shown in mmol/L.

FIG. 2 shows the quenched ADPI (APDI 50 μmol/L+Cu 2 mmol/L) responds proportionately from 0 to 12 mmol/L PPi in both HEPES buffer and saliva. The background concentration of PPi in saliva is 2.5 mmol/L in this case.

FIG. 3 shows the color difference between (a) APDI-Cu in saliva, (b) APDI-Cu in saliva with added PPi and difference in fluorescence (c) APDI-Cu in saliva, (d) APDI-Cu in saliva with added PPi.

FIG. 4 shows (a) Fishburne tab; (b) tab inserted into the mouth to collect saliva.

FIG. 5 shows an embodiment of a laboratory scale modified RT-LAMP assay. The APDI-Cu dye is used, no initial denaturing heating at 95° C. is performed.

FIGS. 6 and 7 illustrate embodiments of a portable modified RT-LAMP assay.

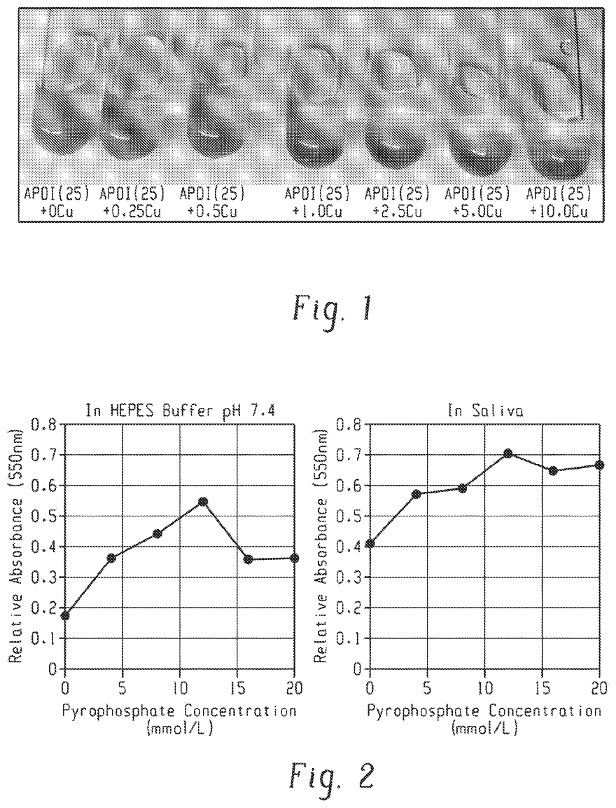

The above-described and other features will be appreciated and understood by those skilled in the art from the following detailed description, drawings, and appended claims.

### DETAILED DESCRIPTION

Described herein is a modified nucleic acid amplification and detection technology, e.g., an RT-LAMP technology, that makes possible the use of saliva for the detection of viruses such as SARS-CoV-2 with high selectivity and specificity in a portable (non-laboratory) test kit. These modifications also allow for the elimination of high temperature cycles, and therefore the method does not require laboratory support for the analysis. A portable test kit is also described.

Specifically, an indicator that responds to pyrophosphate, an amplification reaction product, has been identified. Advantageously, the indicator includes complexed copper which not only provides a detectable color change but also inhibits unwanted enzymes in the sample (e.g., saliva). The methods described herein provide for the analysis of a wide variety of samples including saliva in a portable kit.

Particular to the use of saliva samples, prior art RT-LAMP assays are limited by the detection method. When a pH indicator is used to detect DNA amplification, if the sample has a low pH or high buffer capacity, then the test could indicate a false positive result. When a magnesium indicator is used, if the sample has significant concentrations of Mg, Ca or other metals, these other metals can interfere with the magnesium indicators also resulting in false positive results. In addition, RNAse or DNA polymerase inhibitors can provide false negative results. RNase is an enzyme that destroys RNA. If present at the time the virus particles are lysed, then the viral RNA can be attacked before it can be transcribed into DNA. RNase is common in many biological samples. Therefore, samples are typically treated to eliminate RNase prior to adding RT-LAMP reagents. In addition, DNA polymerase is a component of the RT-LAMP reagents and is needed to copy the virus-derived DNA.

Pre-analysis strategies to overcome these issues have included heating at 95° C. to denature matrix RNase, adding base to establish the pH in the optimal range, and dilution of the sample to lower its buffer capacity. However, these protocols can be challenging to implement in a non-laboratory setting. Elevated temperature generally requires laboratory equipment. If the reagents are pH-sensitive, pH must be controlled starting with the sample. Also, the DNA amplification reaction generates H⁺ ions and the pH of the solution may change by as much as 3 pH units. If the indicator for other reaction products is pH sensitive, then there will be false readings.

In addition, it is preferred that the sample be accessible, have a homogeneous composition and avoid contamination. Blood samples are highly invasive to collect, have a low virion level and suffer from immune system interference, and may have high concentrations of RNase. Nasal swabs are also highly invasive and inconsistent, are non-homogeneous, and mucus-coated virions may not lyse during protocols. Saliva, however, is easily accessible and has a high virion count. However, saliva also has a variable pH and RNase concentration which, as explained above, can confound results in prior art protocols.

The modified nucleic acid amplification technology described herein solves many of these issues.

In an RNA amplification reaction such as an RT-LAMP reaction, the viral RNA is transcribed into DNA. DNA segments that match the virus specific primers are copied. The DNA amplification is accomplished using ATP to add nucleotides to the DNA. The DNA polymerase reaction produces H⁺ ions and pyrophosphate as products of the amplification. A new indicator for pyrophosphate (PPi) has been identified that is highly selective and can be used to evaluate the outcome of the RT-LAMP reactions.

The indicator is based on perylene-3,4:9,10-tetracarboxylic dianhydride (PDI) an industrial dye called Pigment Red 224 that is functionalized with amino acids and complexed with copper ions (AAPDI-Cu). PDI is insoluble in H₂O, however it can be made soluble in H₂O through functionalization with amino acids (e.g., aspartic acid, glycyl-1-aspartic acid, glutamic acid, etc.) as shown in Scheme 1. The aspartic-PDI (APDI) dye is red in color.

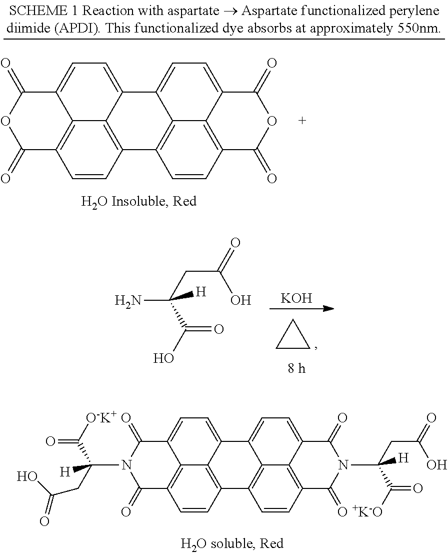

The amino acid groups are able to complex copper as shown in Scheme 2. The red color of the APDI is quenched to a pink color when complexed with Cu.

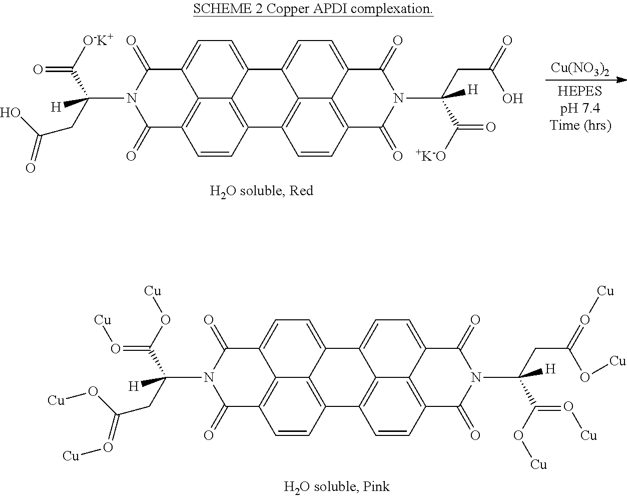

As shown in Scheme 3, the pyrophosphate specific reaction: Cu is highly selective and only binds with PPi.

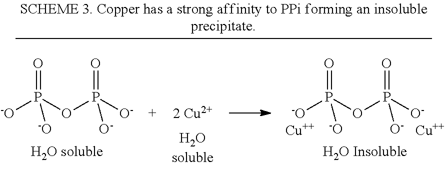

As shown in the art, the selectivity for copper over other cations includes Li, Na, K, Mg, Fe, Co, Ni, Zn, and Ag which do not bind with PPi as strongly as with copper. (Dey et. al, ACS Omega, 4, pp. 16191-16200, 2019). Relevant to the methods described herein, PPi generated by RT-LAMP DNA amplification removes the Cu from APDI-Cu complex; free APDI is red. See Scheme 4.

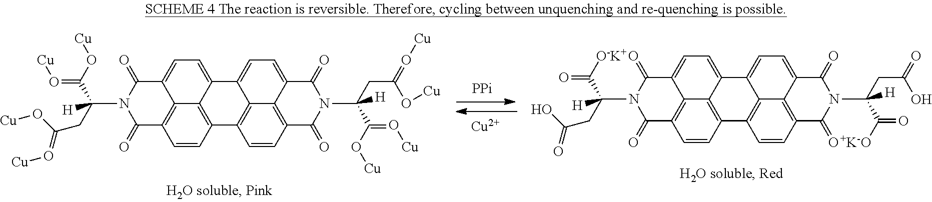

Other important features of the methods include the observation that Cu inhibits the activity of RNase which otherwise destroys virion RNA as the virions are disrupted. The use of copper may thus eliminate the need for the first 95° C. heating step in the current methods. The use of Cu is also expected to lower the temperature needed for the amplification steps. Advantageously, Cu ions do not inhibit DNA polymerase. Also, ADPI is insensitive to pH over a range of pH 5-9. Further, APDI is highly fluorescent under UV light to give more definitive results, quenched APDI-Cu is not fluorescent.

In an aspect, a modified method for detecting the presence or absence of a target nucleic acid comprises contacting a sample suspected of containing the target nucleic acid with one or more primer sets, divalent ions, dNTPs, a buffer, a polymerase, and an amino-acid functionalized perylene-3,4:9,10-tetracarboxylic dianhydride (PDI) dye complexed with copper (X-PDI-Cu) to form an amplification mixture, wherein amino acid X is selected from aspartic acid, glycyl-1-aspartic acid, and glutamic acid; incubating the amplification mixture under conditions to perform an amplification reaction providing amplified target nucleic acid and pyrophosphate; and detecting uncomplexed X-PDI in the reaction mixture, wherein uncomplexed X-PDI indicates the production of Cu-pyrophosphate and the presence of the target nucleic acid in the sample.

In general, nucleic acid amplification techniques utilize enzymes (e.g. polymerases) to generate copies of a target nucleic acid that is bound specifically by one or more oligonucleotide primers. Non-limiting examples of amplification techniques include one or more of the polymerase chain reaction (PCR), the reverse transcription polymerase chain reaction (RT-PCR), strand displacement amplification (SDA), helicase dependent amplification (HDA), recombinase polymerase amplification (RPA), loop-mediated isothermal amplification (LAMP), rolling circle amplification (RCA), transcription-mediated amplification (TMA), self-sustained sequence replication (3SR), and nucleic acid sequence based amplification (NASBA).

Loop-mediated isothermal amplification (LAMP) is a method of amplifying target nucleic acids with high sensitivity and specificity under isothermal conditions. The LAMP method includes a standard method, in which a DNA polymerase having a strand displacement activity and a primer set consisting of at least four or six primers specific to the several regions of target nucleic acids are used as a primer set.

PCR relies on a thermostable DNA polymerase, e.g., Taq polymerase, and uses DNA primers designed specifically for the DNA region of interest. In PCR, the reaction is repeatedly cycled through a series of temperature changes, which allow many copies of the target region to be produced. RT-PCR includes a reverse transcription reaction.

Strand displacement amplification (SDA) is an isothermal, in vitro nucleic acid amplification technique based upon a restriction endonuclease such as HincII nicking its recognition site and a polymerase extending the nick at its 3′ end, which displaces the downstream strand. For example, HincII can nick the unmodified strand of a hemiphosphorothioate form of its recognition site. Exponential amplification results from coupling sense and antisense reactions in which strands displaced from a sense reaction serve as target for an antisense reaction and vice versa. ST-SDA can also be employed.

Helicase dependent amplification (HDA) is an isothermal in vitro nucleic acid amplification technique. Strands of double stranded DNA are first separated by a DNA helicase and coated by single stranded DNA (ssDNA)-binding proteins. DNA polymerase is then used to extend the sequence-specific primers annealed to the templates to produce a double stranded DNA, and the two newly synthesized DNA products are then used as substrates by DNA helicases, entering the next round of the reaction, providing exponential amplification of the selected target sequence. RT-HDA can also be employed.

Recombinase polymerase amplification (RPA) is another isothermal nucleic acid amplification technique. RPA employs a recombinase, a single-stranded DNA binding protein and a strand-displacing polymerase. The recombinase pairs primers with the complementary sequence in the DNA, the single-stranded DNA binding protein prevents the primers from being displaced, and the strand-displacing DNA polymerase synthesizes DNA from where the primer has bound the target nucleic acid. RT-RPA can also be employed.

Rolling circle amplification (RCA) is an isothermal enzymatic process that uses DNA or RNA polymerases to synthesize multiple copies of circular nucleic acids. In RCA, circular template is ligated, primer-induced single stranded elongation is performed off the circular template, and the amplified product is then detected. Multiple primers can be used to produce multiple RNA products.

Transcription-mediated amplification (TMA) is an isothermal nucleic acid amplification system. It uses the function of an RNA polymerase to make RNA from a promoter engineered in the primer region, and a reverse transcriptase, to produce DNA from the RNA templates. For TMA, the reverse transcriptase itself degrades the initial RNA template as it synthesizes its complementary DNA. Rolling circle reverse transcription mediated RNA amplification can also be performed.

Self-sustained sequence replication (3SR) is an isothermal transcription based amplification system consisting of continuous cycles of reverse transcription and RNA transcription designed to replicate a nucleic acid (RNA-target) using a double-stranded cDNA intermediate. This method requires three enzymatic activities: reverse transcriptase, DNA-dependent RNA polymerase and ribonuclease H.

Nucleic acid sequence based amplification (NASBA) is an isothermal in vitro amplification technique mimicking retroviral RNA replication that does not require thermal cycling. The activities of reverse transcriptase, ribonuclease H (RNase H), and T7 RNA polymerase combine to produce new RNA targets via newly synthesized double-stranded DNA intermediates.

An amplification reaction typically includes divalent ions, such as magnesium which can be used in the form of a salt, such as magnesium acetate, magnesium chloride, or magnesium sulfate. Exemplary buffers include a sodium phosphate buffer, a potassium phosphate buffer, a Tris-HCl buffer, or a Tricine buffer.

As used herein, DNTPS include standard dNTPs as well as nucleotide analogs. The nucleotide analogs may be modified nucleotides or nucleotides that are not found in nature, and may be polymerized either alone or in conjunction with natural nucleotides during DNA synthesis. Specific examples of nucleotide analogues that may be polymerized through Watson-Crick base pairing include substituted purines or pyrimidines, deazapurines, methylpurines, methylpyrimidines, aminopurines, aminopyrimidines, thiopurines, thiopyrimidines, indole, pynrole, 7-deazaguanine, 7-methylguanine, hypoxanthine, pseudocytosines, pseudoisocytosines, isocytosines, isoguanine, 2-thiopyrimidines, 4-thiothymine, 6-thioguanine, nitropyrroles, nitroindoles, and 4-methylindole, without being limited thereto. Nucleotides including substituted deoxyribose analogs include substituted or unsubstituted arabinose, substituted or unsubstituted xylose, and substituted or unsubstituted pyranose. Nucleotides including phosphate ester analogs include alkylphosphonates, such as phosphorothioates, phosphorodithioates, phosphoramidates, phosphoroselenoates, phosphoranilothioates, phosphoraniladates, phosphoramidates, boron phosphates, phosphotriesters, and methylphosphonates.

The DNA polymerase that may be used in a reaction is a polymerase derived from a thermophilic microorganism, in particular, a polymerase lacking a 5′-3′ exonuclease function. Non-limiting examples of the DNA polymerase include the Bacillus stearothermophilus (Bst) DNA polymerase, the Thermus, thermophilus (Tth) DNA polymerase, the Thermus aquaticus (Taq) DNA polymerase, the Thermococcus litoralis DNA polymerase, the Pyrococcus furiosus (Pfu) DNA polymerase, and the Bacillus caldotenax DNA polymerase.

In an aspect, the amplification mixture further comprises manganese, which in addition to copper, can inhibit RNAse activity in the sample.

Also included are methods in which reverse transcription is used, i.e., RT-LAMP. In RT-LAMP, the amplification mixture further comprises a reverse transcriptase. Non-limiting examples of reverse transcriptases that may be used in a reaction include the moloney murine leukemia virus (MMLV) reverse transcriptase and the avian myeloblastosis virus (AMV) reverse transcriptase.

Thus, in an aspect, a DNA polymerase and a reverse transcriptase are used together. A reverse transcription reaction and an amplification reaction may be performed in one reaction, thereby increasing convenience. In particular, when samples, such as saliva, are directly used without isolating nucleic acids, RNA and DNA of viruses are all present in these samples, so that efficient amplification is possible. In the RT-LAMP reaction, since reverse transcribed DNA as well as DNA may be amplified, DNA and/or RNA of proviruses may be amplified.

In an aspect, the amplification reaction, e.g., RT-LAMP, does not include a step of heating to 95° C. In another aspect, the contacting and incubating are performed at a temperature of less than 65° C.

In an aspect, detecting color is fluorescence detection, colorimetric detection, or a combination thereof.

Design of primers for amplification is known in the art and involves choice of target region, design of primer candidates, and routine experimental screening. Optimization of primer concentrations may be tested experimentally and is routine in the art. In some embodiments, the primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art. For example, in some embodiments, primers may be synthesized by chemical synthesis, genetic engineering techniques, and/or artificial manipulation of isolated segments of nucleic acids.

In choosing target pathogen nucleic acids, nucleic acid sequences from pathogen genes can be selected from regions known to maximize inclusivity across known strains, and/or minimize cross-reactivity with related pathogens and genomes likely to be present in the sample. For example, the primers for amplification of SARS-CoV-2 nucleocapsid (N) gene exemplified herein were selected from regions of the virus N gene to maximize inclusivity across known SARS-CoV-2 strains and minimize cross-reactivity with related viruses and genomes likely to be present in the sample. Similarly, other oligonucleotide primers and probes can be selected from SARS-CoV-2 N gene as well as other regions of the SARS-CoV-2 genome, e.g., envelope (E) gene, membrane (M) gene, and/or spike (S) gene.

It should be appreciated that while some examples of the methods provided herein are discussed in the context of specific pathogens or diseases (e.g., SARS-CoV-2), the techniques are not so limited and can be used with any pathogen or disease in which nucleic acid molecules characteristic to or indicative of such pathogen or disease may be detected. Therefore, the examples provided herein of the various embodiments are intended for exemplary purposes only.

In an aspect, the target nucleic acid is from a pathogen, a virus, a bacterium, a protozoan, a prion, a viroid, a parasite, a fungus. Exemplary bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumannii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melitensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetiid, Corynebacterium sp. (such as, Corynebacterium diphtheriae, Corynebacterium jeikeium and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chaffeensis and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingae, Klebsiella sp. (such as Klebsiella pneumoniae, Kiebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia haemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasma sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella choleraesuis and Salmonella typhimurium), Serratia sp. (such as Serratia marcescens and Serratia liquefaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus haemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equisimilis, Group D streptococci, Streptococcus bovis, Group F streptococci, and Streptococcus anginosus Group G streptococci), Spirillum minus, Streptobacillus moniliformis, Treponema sp. (such as Treponema carateum, Treponema pertenue, Treponema pallidum and Treponema endemicum, Trichophyton rubrum, T mentagrophytes, Tropheryma whippelii, Ureaplasma urealyticum, Veillonella sp., Vibrio sp. (such as Vibrio cholerae, Vibrio parahaemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metschnikovii, Vibrio damsela and Vibrio furnissii), Yersinia sp. (such as Yersinia enterocolitica, Yersinia pestis, and Yersinia pseudotuberculosis) and Xanthomonas maltophilia among others.

In another aspect, a target nucleic acid is from a fungus or fungi. Examples of fungi that can be detected in accordance with the disclosed methods include without limitation any one or more of (or any combination of), Aspergillus, Blastomyces, Candidiasis, Coccidioidomycosis, Cryptococcus neoformans, Cryptococcus gattii, sp. Histoplasma sp. (such as Histoplasma capsulatum), Pneumocystis sp. (such as Pneumocystis jirovecii), Stachybotrys (such as Stachybotrys chartarum), Mucormycosis, Sporothrix, fungal eye infections ringworm, Exserohilum, Cladosporium.

In certain aspects, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

In certain aspects, the nucleic acid is from a protozoa. Examples of protozoa that can be detected in accordance with the disclosed methods and devices include without limitation any one or more of (or any combination of), Euglenozoa, Heterolobosea, Diplomonadida, Amoebozoa, Blastocystis, and Apicomplexa. Example Euglenozoa include, but are not limited to, Trypanosoma cruzi (Chagas disease), T. brucei gambiense, T. brucei rhodesiense, Leishmania braziliensis, L infantum, L mexicana, L major, L tropica, and L. donovani. Example Heterolobosea include, but are not limited to, Naegleriafowleri. Example Diplomonadida include, but are not limited to, Giardia intestinalis (G. lamblia, G. duodenalis). Example Amoebozoa include, but are not limited to, Acanthamoeba castellanii, Balamuthia mandrillaris, Entamoeba histolytica. Example Blastocysts include, but are not limited to, Blastocystis hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.

In certain aspects, the nucleic acid is from a parasite. Examples of parasites that can be detected in accordance with disclosed methods include without limitation one or more of (or any combination of), an Onchocerca species and a Plasmodium species.

In an aspect, the target nucleic acid is from a virus. Exemplary viruses include SARS CoV-2, an orthomyxovirus, Hepatitis C Virus (HCV), Ebola virus, influenza, polio, measles, adult Human T-cell lymphotropic virus type 1 (HTLV-1), human immunodeficiency virus (HIV), and Coronavirinae such as OC43, HKU1, 229E, NL63, MERS, SARS-COV, or CCoV, and FCoV.

In an aspect, the target nucleic acid is from a virus. The viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mammarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human parechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Mojiang virus, Mokola virus, Monkeypox virus, Montana myotis leukoencephalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno 116 alophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence.

Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus.

In certain example aspects, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain aspects, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target nucleic acid molecule is part of said pathogen or transcribed from a DNA molecule of said pathogen. For example, the target sequence may be comprised in the genome of an RNA virus.

In certain aspects, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus)

In certain example aspects, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zoster virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Marseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidiovirus, among others Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

When the virus is SARS CoV-2, the one or more primer sets can include a primer set to amplify a portion of a gene encoding an ORFIan replication protein, a primer set to amplify a portion of a gene encoding an envelope protein, and a primer set to amplify a portion of a gene encoding a replication protein nucleocapsid protein.

In an aspect, the sample comprises saliva, tears, blood, throat, nasal, urine, human waste and the other samples which may include viral nucleic acids, for example. A specific sample is a saliva sample.

Also included herein are kits for the detection of nucleic acids. In an aspect, a kit comprises a reagent compartment comprising one or more primer sets to amplify a portion of a target nucleic acid, divalent ions, dNTPs, a buffer, a polymerase, and an amino-acid functionalized perylene-3,4:9,10-tetracarboxylic dianhydride (PDI) dye complexed with copper (X-PDI-Cu), wherein amino acid X is selected from aspartic acid, glycyl-1-aspartic acid and glutamic acid; and a sample collection device for collecting a sample from a subject; wherein, upon contacting the sample with the reagent compartment, an amplification reaction provides amplified target nucleic acid and pyrophosphate. The kit may further comprise instructions for performing the amplification reaction and detecting color of the amplification reaction, wherein the color indicates the production of pyrophosphate in the amplification mixture, and wherein a color change indicated the presence of the target nucleic acid in the sample.

In an aspect, wherein the sample is a saliva sample, the sample collection device comprises an absorbent paper tab, and the kit comprises a housing for contacting the absorbent paper tab with the reagent region.

In an aspect, an exemplary salivary sample collection device is described in U.S. Pat. No. 10,058,307.

In an aspect, the kit includes a negative control and a positive control.

The test methods and kits described herein are highly sensitive and accurate and may be safely and easily operated or conducted by untrained individuals. As a result, the methods and kits may be useful in a wide variety of contexts. For example, in some cases, the methods and kits may be available over the counter for use by consumers. In such cases, untrained consumers may be able to self-administer the test (or administer the test to friends and family members) in their own homes (or any other location of their choosing) without the assistance of another person. In some cases, the diagnostic tests, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the tests or methods may be operated or performed by the test subjects (e.g., students, teachers, patients, employees) or by designated individuals (e.g., a school nurse, a teacher, a school administrator, a receptionist). Point-of-care administration is also contemplated herein, where the diagnostic tests, kits, or methods are administered by a trained medical professional in a point-of-care setting. Certain embodiments additionally contemplate a downloadable software component or software ecosystem, which may assist with test result readout and data aggregation.

As shown in the Examples below, the method and system described herein provides a rapid test which produces results in less than 1 hour with high sensitivity. In some embodiments, the total time for performing the diagnostic method is about 60 minutes or less, about 50 minutes or less, 45 minutes or less, about 40 minutes or less, or about 30 minutes or less, or about 20 minutes or less.

In some embodiments, the methods of the present disclosure are applied to a subject who is suspected of having a pathogenic infection or disease, but who has not yet been diagnosed as having such an infection or disease. A subject may be “suspected of having” a pathogenic infection or disease when the subject exhibits one or more signs or symptoms of such an infection or disease. Such signs or symptoms are well known in the art and may vary, depending on the nature of the pathogen and the subject. Signs and symptoms of disease may generally include any one or more of the following: fever, chills, cough (e.g., dry cough), generalized fatigue, sore throat, runny nose, nasal congestion, muscle aches, difficulty breathing (shortness of breath), congestion, runny nose, headaches, nausea, vomiting, diarrhea, loss of smell and/or taste, skin lesions (e.g., pox), or loss of appetite. Other signs or symptoms of disease are specifically contemplated herein. As a non-limiting example, symptoms of coronaviruses (e.g., COVID-19) may include, but are not limited to, fever, cough (e.g., dry cough), generalized fatigue, sore throat, runny nose, nasal congestion, muscle aches, loss of smell and/or taste, and difficulty breathing (shortness of breath). As a non-limiting example, symptoms of influenza may include, but are not limited to, fever, chills, muscle aches, cough, sore throat, runny nose, nasal congestion, and generalized fatigue. A subject may also be “suspected of having” a pathogenic infection or disease despite exhibiting no signs or symptoms of such an infection or disease (e.g., the subject is asymptomatic). Accordingly, the methods disclosed herein can be adapted for use in other methods (or in combination) with other methods that require quick identification of pathogen species, monitoring the presence of pathogen proteins (antigens), antibodies, antibody genes, detection of certain phenotypes (e.g., bacterial resistance), monitoring of disease progression and/or outbreak, and antibiotic screening. Because of the rapid and sensitive diagnostic capabilities of the embodiments disclosed here, detection of pathogen species type, down to a single nucleotide difference, and the ability to be deployed as a POC device, the embodiments disclosed herein may be used to guide therapeutic regimens, such as selection of the appropriate antibiotic or antiviral. The embodiments disclosed herein may also be used to screen environmental samples (air, water, surfaces, food etc.) for the presence of microbial contamination.

The invention is further illustrated by the following non-limiting examples.

### EXAMPLES

### Methods

### Example 1: Proof of Concept

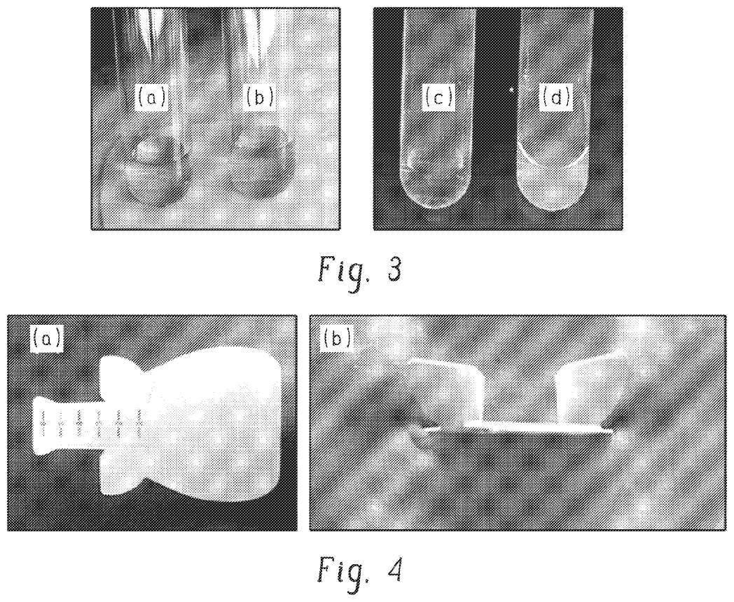

When the APDi-Cu dye is used in a LAMP reaction, the red color is proportional to the concentration of PPi generated by RT-LAMP DNA amplification as shown in FIG. 2. As shown in FIG. 3, the color change is sufficient to distinguish between no PPi and PPi at RT-LAMP concentrations. Additionally, the change in fluorescence is sufficient to distinguish between no PPi and PPi at RT-LAMP concentrations.

### Example 2: Design of Saliva Collection System

Saliva collection by expectoration into a tube raises concerns of aerosol formation and of sample handling. Saliva collected in a tube for instance should be carefully collected so that no saliva is on the outside of the tube, and that the tube and sample should be heat sterilized prior to opening of the tube.

Direct collection of saliva by a swab which is then placed into a container requires significant care as the sample volume is typically small which can lead to inconclusive test results. Additionally, the swab sample may not be homogeneous depending on how it was collected.

We propose collection of saliva on an absorbent tab that was originally designed for assessment of xerostomia. This device has been approved by the FDA for saliva collection. It collects about 150 μL of saliva. The absorbent system tends to buffer the pH to 7.5-8.0. FIG. 4 shows an absorbent tab and the absorbent tab inserted into the mouth to collect saliva.

### Example 3: Laboratory Scale Modified RT-LAMP Assay

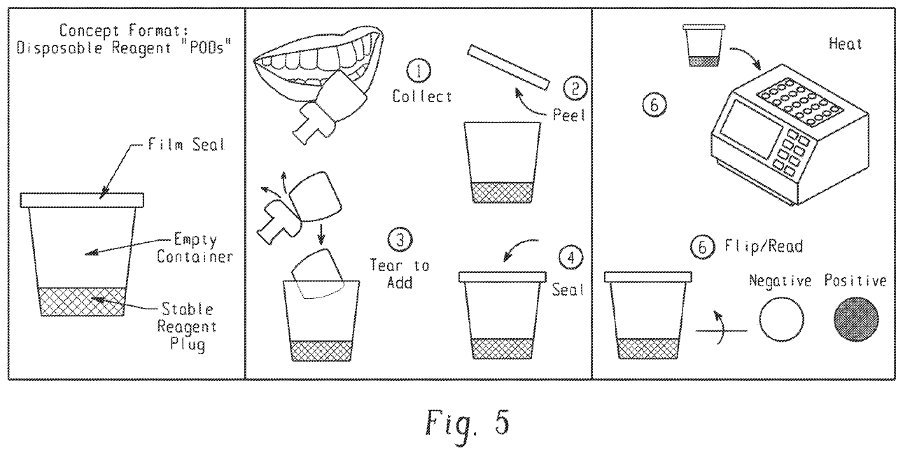

FIG. 5 illustrates a laboratory scale test. The Fishburne tab can be placed in a holder or container that contains the RT-LAMP reagents. The APDI-Cu dye is used, no initial denaturing heating at 95° C. is performed. In this proof of concept assay, a small container has a gel containing all of the modified RT-LAMP reagents. The saliva is collected using the Fishburne tab placed on the tongue for 3 seconds. The tab is inserted face up into the container which is resealed. The container is then placed in a 65° C. environment which melts the gel releasing the reagents to react with the saliva on the tab. After 20 minutes, the container is cooled on ice and the gel reforms. Flip the container to determine the test result; pink is negative, red is positive for COVID-19.

### Example 4: Portable Assay Kit

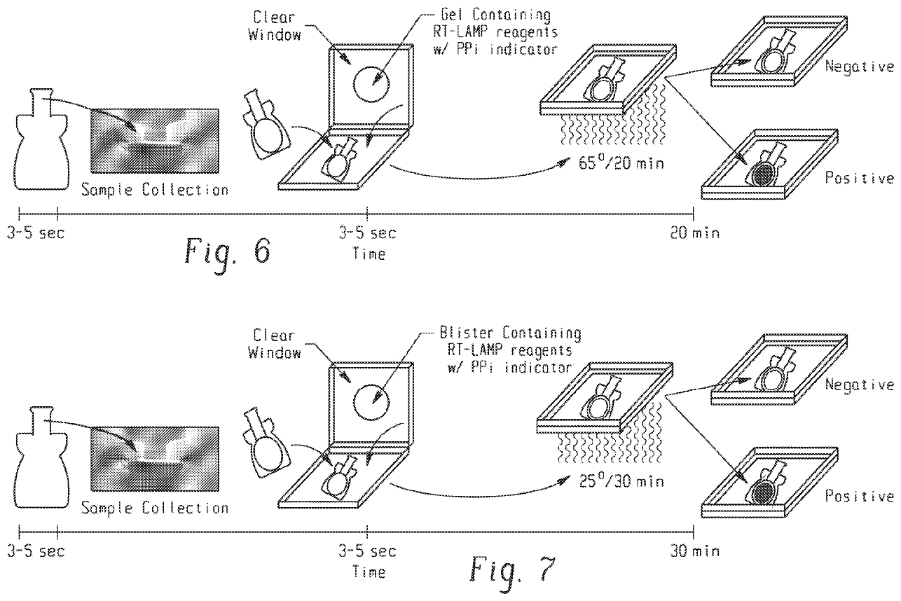

In a portable assay kit as illustrated in FIG. 6, the Fishburne tab is placed in a portable kit. The saliva sample is collected on the Fishburne tab (3 seconds) and transferred to a closable box that has a clear lid. The modified RT-LAMP reagents are in a gel attached to the inner surface of the box top. The box is closed and placed on a 65° C. surface such that the gel melts releasing the reagents onto the sample. After 20 minutes the results can be read without opening the kit.

In another aspect as illustrated in FIG. 7, a portable kit is designed where no heating of the sample is required. The time for results to develop may be longer due to the lack of a heating step.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within t 10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A modified method for detecting the presence or absence of a target nucleic acid, the method comprising
contacting a sample suspected of containing the target nucleic acid with one or more primer sets to amplify a portion of the target nucleic acid, divalent ions, dNTPs, a buffer, a polymerase, and an amino-acid functionalized perylene-3,4:9,10-tetracarboxylic dianhydride (PDI) dye complexed with copper (X-PDI-Cu) to form an amplification mixture, wherein amino acid X is selected from aspartic acid, glycyl-1-aspartic acid, and glutamic acid;
incubating the amplification mixture under conditions to perform an amplification reaction providing amplified target nucleic acid and pyrophosphate; and
detecting uncomplexed X-PDI in the reaction mixture, wherein uncomplexed X-PDI indicates the production of Cu-pyrophosphate and the presence of the target nucleic acid in the sample.

2. The method of claim 1, wherein the amplification reaction is a polymerase chain reaction (PCR), a reverse transcription polymerase chain reaction (RT-PCR), a strand displacement amplification (SDA), a helicase dependent amplification (HDA), a Recombinase Polymerase Amplification (RPA), a reverse transcription loop-mediated isothermal amplification (RT-LAMP), a rolling circle amplification (RCA), a transcription-mediated amplification (TMA), a self-sustained sequence replication (3SR), or a nucleic acid sequence based amplification (NASBA).

3. The method of claim 1, wherein the amplification mixture further comprises manganese.

4. The method of claim 1, wherein the amplification reaction is an RT-LAMP reaction and the amplification mixture further comprises a reverse transcriptase.

5. The method of claim 4, wherein the method does not include a step of heating to 95° C.

6. The method of claim 4, wherein contacting and incubating are performed at a temperature of less than 65° C.

7. The method of claim 1, wherein detecting is fluorescence detection, colorimetric detection, or a combination thereof.

8. The method of claim 1, wherein the target nucleic acid is from a virus, a bacterium, a fungus, or a protozoan.

9. The method of claim 8 wherein the virus is selected from SARS CoV-2, an orthomyxovirus, Hepatitis C Virus (HCV), Ebola virus, influenza, polio, measles, adult Human T-cell lymphotropic virus type 1 (HTLV-1) and human immunodeficiency virus (HIV), OC43, HKU1, 229E, NL63, MERS, SARS-COV, CCoV, and FCoV.

10. The method of claim 9, wherein the virus is SARS CoV-2, and wherein the one or more primer sets includes a primer set to amplify a portion of a gene encoding an ORF a replication protein, a primer set to amplify a portion of a gene encoding an envelope protein, and a primer set to amplify a portion of a gene encoding a replication protein nucleocapsid protein.

11. The method of claim 1, wherein the sample is a salivary sample, blood, throat, nasal, tears, urine, or human waste, specifically a salivary sample.

12. A modified method for detecting the presence or absence of SARS CoV-2, the method comprising
contacting a sample suspected of containing SARS CoV-2 with one or more primer sets to amplify a portion of the SARS CoV-2 genome, divalent ions, dNTPs, a buffer, a polymerase, and an amino-acid functionalized perylene-3,4:9,10-tetracarboxylic dianhydride (PDI) dye complexed with copper (X-PDI-Cu) to form an amplification mixture, wherein amino acid X is selected from aspartic acid, glycyl-1-aspartic acid and glutamic acid;
incubating the amplification mixture under conditions to perform an amplification reaction providing amplified SARS CoV-2 nucleic acids and pyrophosphate; and
detecting uncomplexed X-PDI in the reaction mixture, wherein uncomplexed X-PDI indicates the production of Cu-pyrophosphate and the presence of the SARS CoV-2 nucleic acids in the sample.

13. The method of claim 12, wherein the virus is SARS CoV-2, and wherein the one or more primer sets includes a primer set to amplify a portion of a gene encoding an ORFIan replication protein, a primer set to amplify a portion of a gene encoding an envelope protein, and a primer set to amplify a portion of a gene encoding a replication protein nucleocapsid protein.

14. The method of claim 12, wherein the sample is a salivary sample, blood, throat, nasal, tears, urine, or human waste, specifically a salivary sample.

15. The method of claim 12, wherein the amplification reaction is an RT-LAMP reaction, and the amplification mixture further comprises a reverse transcriptase.

16. The method of claim 15, wherein the method does not include a step of heating to 95° C.

17. The method of claim 15, wherein contacting and incubating are performed at a temperature of less than 65° C.

18. The method of claim 12, wherein detecting color is fluorescence detection, colorimetric detection, or a combination thereof.

19. A kit comprising,
a reagent compartment comprising one or more primer sets to amplify a portion of a target nucleic acid, divalent ions, dNTPs, a buffer, a polymerase, and an amino-acid functionalized perylene-3,4:9,10-tetracarboxylic dianhydride (PDI) dye complexed with copper (X-PDI-Cu), wherein amino acid X is selected from aspartic acid, glycyl-1-aspartic acid and glutamic acid; and
a sample collection device for collecting a sample from a subject;
wherein, upon contacting the sample with the reagent compartment, an amplification reaction provides amplified target nucleic acid and pyrophosphate.

20. The kit of claim 19, wherein the sample is a saliva sample, the sample collection device comprises an absorbent paper tab, and the kit comprises a housing for contacting the absorbent paper tab with the reagent region.

21. The kit of claim 19, wherein the kit includes a positive control and a negative control.
